(19) United States
(12) Patent Application Publication
(10) Pub. No.: US 2004/0167622 A1
(43) Pub. Date: Aug. 26, 2004

(54) Temporary refractive intraocular lens and methods of use

(76) Inventors: Murad A. Sunalp, Visalia, CA (US); Nino Sorgente, Houston, TX (US)

Correspondence Address: DONN K. HARMS, PATENT & TRADEMARK LAW CENTER, SUITE 100, 12702 VIA CORTINA, DEL MAR, CA, 92014, US

(21) Appl. No.: 10/398,996

(22) Filed: Apr. 4, 2003

Related U.S. Application Data
(63) Continuation of application No. PCT/US01/09443, filed on Mar. 26, 2001.

(51) Int. Cl. A61F 2/16; A61F 9/00

(52) U.S. Cl. 623/6.12; 623/907

(57) ABSTRACT

The invention describes a temporary intraocular lens for use in intraocular lens replacement therapy, and uses of the temporary lens for determining the refractive status of the eye in patients having lens replacement therapy. Also described are methods for manufacturing a temporary intraocular lens.

### TECHNICAL FIELD

The present invention relates generally to manufacture and use of temporary intraocular lenses for the accurate measurement of the refractive status of the eye of patients before implantation of permanent intraocular lenses. The temporary intraocular lens can be manufactured of any clear refractive material, and of any size that will fit the surgical incision.

### BACKGROUND OF THE INVENTION

Since the first successful intraocular lens implant by Ridley in 1949, advances in IOL implants have led to the routine replacement of the lens with an IOL following cataract surgery, as well as in other interventions requiring lensectomy. Originally intraocular lenses were all of the same power, but as technology has advanced, intraocular lenses of different powers have been produced to fit individual patients with a lens that gives optimal visual acuity. In addition, a number of materials and designs have been developed as well as surgical techniques, which give the surgeon a wide choice of options.

For optimal refractive outcome the power of the IOL to be implanted is calculated using various formulas (Holladay, J T. J. Cataract Refract Surg. 23:1356-1370, 1997). These various formulas require that a number of parameters be known; these include axial length, corneal power (keratometry), preoperative refractive error, desired postoperative refractive error, vertex distance, and anterior chamber depth. Even though various methods have been developed to measure these parameters (Hoffer, K J. 20:55-562, 1994; Holladay, J T and Prager, T C. Am. J. Ophthalmol. 107:189-190, 1989) errors do occur which will lead to errors in the desired refractive outcome. For example, an error of 1 mm in the measured axial length will result in an error in the calculation of the IOL power of approximately 2.5 D in normal eyes; in long eyes (30 mm) the error is approximately 1.75 D and in short eyes (20 mm) the error is approximately 3.75 D. In addition, it is not possible to obtain accurate measurements of corneal power in cases where previous corneal surgery changes the original keratometry readings or in cases where the cornea is scarred. Even though special formulas have been developed to calculate IOL power for eyes after refractive surgery (Hoffer, K J. J. Refract. Surg. 11:490-493, 1995), for phakic eyes (Holladay, J T. Am. J. Ophthalmol. 11:63-66, 1993), these formulas do not predict the IOL power accurately, and significant errors in the desired refractive outcome can occur, especially in patients with atypical ocular dimensions, in patients who had previous refractive surgery, and in patients with an uneven corneal surface.

Therefore it would be desirable to provide a means that would accurately determine the refractive status of the operated eye. The present invention is intended for the manufacture and use of a temporary refractive intraocular lens to calculate the exact power of the permanent intraocular lens.

### BRIEF SUMMARY OF THE INVENTION

The present invention describes a temporary intraocular lens and uses thereof for accurate measurement of the refractive status of the eye in patients during an intraocular lens replacement procedures. A temporary intraocular lens of this invention is used to calculate the exact power of the permanent intraocular lens.

In one embodiment, the invention describes a method for intraocular refraction of a patient during a lens replacement surgical procedure comprising:

(a) inserting a temporary intraocular lens into the eye of a patient;

(b) refracting the eye to determine the refractive status of the eye; and

(c) removing the temporary intraocular lens.

The lense can be of any suitable size, typically, a size appropriate to a surgical incision in said surgical procedure.

In preferred embodiments, the lens used in the method comprises a means for manipulation of the lens during said procedure, to facilitate the placement and removal of the temporary lens.

Thus in a related embodiment, the invention contemplates a temporary intraocular lens comprising a lens and a means for manipulation of the lens during a temporary intraocular implant and refraction procedure. In one embodiment, the means for manipulation is an indentation in the lens adapted to conform to a tool for retrieval of the lens following refraction of the temporary lens. Typically, the indentation is one or more holes. Alternatively, the means for manipulation can be an appendage to facilitate gripping the lens for removal. An exemplary appendage is an attached suture, a leash, a tab, a loop, or other appendage, as is well known.

Preferably, the lens comprises a material that can be sterilized. A typical lens is comprised of a clear refractive material, and can be a clear rigid plastic, a clear soft plastic, and the like materials. The clear refractive material can be clear silicon, clear collagenous material, a clear chitinous material, or the like materials.

In a further related embodiment, the invention contemplates a method for manufacturing a temporary intraocular lens comprising incorporating a means for manipulation of the temporary intraocular lens during a temporay intraocular implant and refraction procedure. The lens is typically comprised of a material that can be sterilized. The lens can be produced by molding, cutting, lathing or shaping a refractive material into a lens.

### BRIEF DESCRIPTION OF THE DRAWINGS

FIG. 1 is a schematic diagram of certain features of a temporary intraocular lens of the present invention. The small four circles illustrate exemplary positions of hole-type indentations in the lens body to facilitate manipulation and positioning of the lens. The vertical line illustrates a suture attached to the lens and used to remove the lens.

FIG. 2 is a schematic diagram of certain features of a temporary intraocular lens of the invention. The lens shown is oval-shaped, in comparison to a circular shaped lens shown in FIG. 1.

FIG. 3 is a schematic diagram of certain features of a lens of the invention. The lens is shown with three indentations and a loop-type leash attached to the lens body to facilitate manipulation and removal of the lens from the eye.

FIG. 4 is a schematic representation of the structure of the eye during the practice of a intraocular lens replacement method of the invention. Panel A illustrates the aphakic eye following lens removal, including the features of the iris, cornea, zonules and lens capsular bag. Panel B illustrates the inserted temporary refractive intraocular lens, and Panel C illustrates the structure of a permanent intraocular lens in an eye following the removal of the temporary lens and replacement by the permanent lens after refraction with the temporary lens.

### DETAILED DESCRIPTION OF THE INVENTION

The invention describes the manufacture and use of a temporary intraocular lens to be used to obtain accurate measurements of the refractive status of an eye in which a permanent intraocular lens is to be implanted or not to be implanted. The temporary lens can be manufactured using any refractive material, that can be molded, cut, lathed or otherwise shaped. The temporary lens can be of various powers, plus, neutral and negative diopters. Preferably the temporary lens will be manufactured of a material and process that will result in an inexpensive lens that can be disposable. Preferably the temporary lens will be manufactured of a material and process that will result in a lens that can be easily sterilized and packaged, preferably individually.

Insofar as the present invention is not directed to the manufacture of intraocular lenses in general terms, which art is well known, those details are not discussed in detail.

The manufacture of the lens in the present invention can be of plastic or glass or any clear refractive material, it can be rigid or foldable. With the present technology of phacoemulsification the size of the temporary lens can be as small as necessary to obtain an accurate refractive status and as large as will fit in the surgical wound

The shape of the lens can vary. Whatever is the shape of the lens it can have one or more holes to aid in the positioning of the lens; it can have an attached suture, or other appropriate appendages to aid in the implantation or removal of the lens upon completion of refraction. The appendages can be of a material appropriate to the manufacturing process used, it can be attached with a knot, it can be molded into the lens during manufacture or it can be part of the lens material.

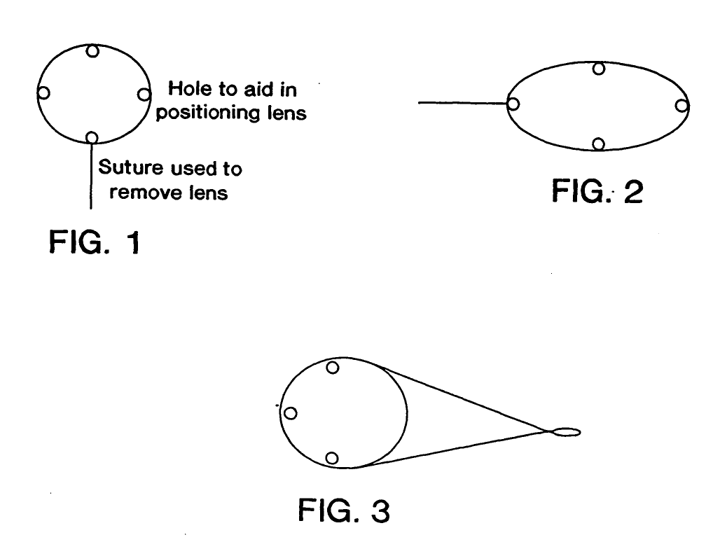

The shapes of lenses shown in FIGS. 1, 2 and 3 are exemplary of the lenses of this invention. These shapes are illustrative only and are not intended to limit the scope of this invention and should not so be construed.

In practicing the present invention, there is provided a method for the calculation of intraocular lens power in cases where the axial length of the eye is too short or too long to obtain accurate measurements. The present invention is also useful for the calculation of intraocular lens power in cases where keratometry readings are difficult to obtain because of surface imperfections, or previous corneal surgery. By using the information obtained by a temporary lens implant and subsequent refraction of the eye, one can determine the refractory status of the eye, and further provide a more accurate description of the desired permanent lens to be used in completing the lens replacement surgery.

### EXAMPLES

### Example 1

Following cataract surgery by phacoemulsification a patient that had been implanted with an IOL of insufficient power three months earlier. The usual procedure to correct for the insufficient power of the IOL, the eye is refracted and a an additional lens is placed on top of the original lens, referred to as a piggyback IOL using a formula such as Gill's.

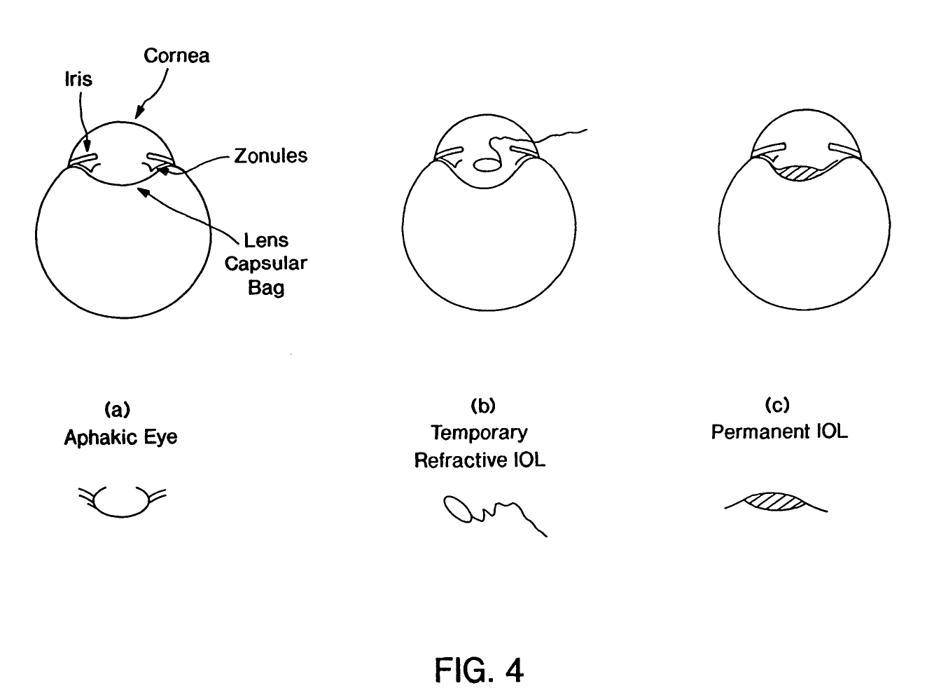

A 3 mm in diameter was cut with a trephine from a foldable acrylic IOL of power+6 diopters and used to refract the eye of a patient as a temporary IOL. The patient was placed in a supine position and a Snellen chart was taped to the ceiling of the operating room directly above the head. The patient was refracted preoperatively, intra-operatively and post op with the permanent IOL in the eye. Intra-operative refraction was done by placing the 3 mm temporary IOL in the anterior chamber, just above the previously implanted IOL, through a 3 mm clear cornea trap door incision made with a 3 mm diamond keratome. Surgery was done with topical anaesthesia so the patient was able to read the Snellen chart. Objective refraction was also done preoperatively, intra-operatively and post op in a similar fashion with retinoscopy. The patient read 20/20 at (adjusted 20 ft Snellen chart) and retinoscopy was −1.00+1.00×90 and as the temporary IOL was anterior to the sulcus position (see FIG. 4) a higher +7.00 permanent foldable IOL was chosen for implantation. Immediately after IOL implantation in the sulcus refraction gave 20/20 uncorrected vision and retinoscopy readings of −1.00+1.00×90 degrees at an intraocular pressure of approximately 20 mm of Hg (measured with operative applanation). The following day the patient had a spherical equivalent of −0.025 and uncorrected vision of 20/20. This lens can also be used primarily after cataract extraction to ascertain the refraction of the eye before implanting the permanent IOL.

### Example 2

Following phacoemulsification, and before removing the viscoelastic material, a temporary refractive IOL is positioned in the capsule appropriately (where the permanent IOL will be positioned), the eye is refracted objectively by retinoscopy and subjectively by placing the patient in a supine position and by taping a Snellen chart to the ceiling of the operating room directly above the patient's head to allow the patient to read the chart. The patient was refracted preoperatively, intra-operatively and post op with the permanent IOL in the eye. A permanent lens of power obtained by refraction with the temporary IOL is chosen and implanted in the patient. A diagrammatic representation of this procedure is shown in FIG. 4.

Although the foregoing has been described in some details by way of illustration and example, for the purpose of clarity and understanding, it is obvious that certain changes and modifications may be practiced within the scope of the appended claims.

What is claimed is:

1. A method for intraocular refraction of a patient during a lens replacement surgical procedure comprising:
(a) inserting a temporary intraocular lens into the eye of a patient;
(b) refracting the eye; and
(c) removing the temporary intraocular lens.

2. The method of claim 1 wherein said lens is of a size appropriate to a surgical incision in said surgical procedure.

3. The method of claim 1 wherein said lens comprises a material that can be sterilized.

4. The method of claim 1 wherein said lens comprises a clear refractive material.

5. The method of claim 4 wherein said clear refractive material comprises a clear rigid plastic.

6. The method of claim 4 wherein said clear refractive material comprises a clear soft plastic.

7. The method of claim 4 wherein said clear refractive material comprises a clear silicon.

8. The method of claim 4 wherein said clear refractive material comprises a clear collagenous material.

9. The method of claim 4 wherein said clear refractive material comprises a clear chitinous material.

10. The method of claim 1 wherein said lens comprises a means for manipulation of the lens during said procedure.

11. The method of claim 1 wherein said procedure further comprises removal of a previously implanted lens prior to inserting said temporary lens.

12. The method of claim 1 wherein said procedure further comprises inserting a permanent intraocular lens following removal of said temporary lens.

13. A temporary intraocular lens comprising a lens and a means for manipulation of the lens during a temporary intraocular implant and refraction procedure.

14. The lens of claim 13 wherein said means for manipulation is an indentation in said lens adapted to conform to a tool for retrieval of said lens.

15. The lens of claim 14 wherein said indentation is one or more holes.

16. The lens of claim 13 wherein said means for manipulation is an appendage.

17. The lens of claim 16 wherein said appendage is an attached suture.

18. The lens of claim 16 wherein said appendage is a leash, a tab or a loop.

19. The lens of claim 13 wherein said lens is of a size appropriate to a surgical incision in said surgical procedure.

20. The lens of claim 13 wherein said lens comprises a material that can be sterilized.

21. The lens of claim 13 wherein said lens comprises a clear refractive material.

22. The lens of claim 21 wherein said clear refractive material comprises a clear rigid plastic.

23. The lens of claim 21 wherein said clear refractive material comprises a clear soft plastic.

24. The lens of claim 21 wherein said clear refractive material comprises a clear silicon.

25. The lens of claim 21 wherein said clear refractive material comprises a clear collagenous material.

26. The lens of claim 21 wherein said clear refractive material comprises a clear chitinous material.

27. A method for manufacturing a temporary intraocular lens comprising incorporating a means for manipulation of the temporary intraocular lens during a temporary intraocular implant and refraction procedure.

28. The method of claim 27 wherein said means for manipulation is an indention in said lens adapted to conform to a tool for retrieval of said lens.

29. The method of claim 28 wherein said indentation is one or more holes.

30. The method of claim 27 wherein said means for manipulation is an appendage.

31. The method of claim 30 wherein said appendage is an attached suture.

32. The method of claim 30 wherein said appendage is a leash, a tab or a loop.

33. The method of claim 27 wherein said lens is of a size appropriate to a surgical incision in said surgical procedure.

34. The method of claim 27 wherein said lens comprises a material that can be sterilized.

35. The method of claim 27 wherein said lens comprises a clear refractive material.

36. The method of claim 35 wherein said clear refractive material comprises a clear rigid plastic.

37. The method of claim 35 wherein said clear refractive material comprises a clear soft plastic.

38. The method of claim 35 wherein said clear refractive material comprises a clear silicon.

39. The method of claim 35 wherein said clear refractive material comprises a clear collagenous material.

40. The method of claim 35 wherein said clear refractive material comprises a clear chitinous material.

41. The method of claim 27 wherein said lens is produced by molding, cutting, lathing or shaping a refractive material into a lens.
